(19) United States
(12) Patent Application Publication
(10) Pub. No.: US 2016/0183898 A1
(43) Pub. Date: Jun. 30, 2016

(54) A MAMMOGRAPHIC DEVICE

(71) Applicant: CIRDAN IMAGING LIMITED, Lisburn Antrim (GB)

(72) Inventors: HUGH CORMICAN, LISBURN (GB); SEAN LATIMER, LISBURN (GB); STEPHEN DUNNIECE, LISBURN (GB)

(21) Appl. No.: 14/910,497

(22) PCT Filed: Aug. 5, 2014
(86) PCT No.: PCT/EP2014/066863

§ 371 Date: Feb. 5, 2016

(30) Foreign Application Priority Data
Aug. 5, 2013 (GB) 1313994.4

(51) Int. Cl. A61B 6/00; A61B 6/04

(52) U.S. Cl. CPC A61B 6/502; A61B 6/0435; A61B 6/0414

(57) ABSTRACT

A mammographic device having a support arrangement for carrying a mammographic head movably mounted on the support arrangement. The mammographic head has a breast clamping assembly, wherein the mammographic head is movably mounted on the support arrangement so as to be movable in response to forces transmittable through the breast clamping assembly via a patient's clamped breast as a result of the patient's body movements.

The present invention relates to a mammographic device and in particular to a mammographic biopsy device with improved articulation relative to a patient's body.

There are currently two basic designs for stereotactic breast biopsy systems. The first system is a dedicated prone table arrangement where the patient lies prone on the table and the affected breast is pendulantly suspended through an aperture in the table. The breast is positioned against an image receptor and held in position by a compression paddle clamping the breast against the image receptor. This type of breast biopsy system enables the stereotactic biopsy team to perform the procedure beneath the table, advantageously out of the patient's view. The second type of prior art arrangement is an upright add on unit where a stereotactic biopsy unit is added onto a standard mammography unit converting it to a stereotactic biopsy system. This equipment allows the biopsy to be performed while the patient is seated, standing or lying in the lateral decubitus position.

In the existing prone table arrangement and the upright add on unit, a patient's body is the only non controllable movable physical feature in the overall system. All of the other components are fixed in position relative to one another so that patient movement will not cause movement of the equipment during the critical stages of imaging and invasive procedure. While setting the equipment up for imaging and for invasive procedures the components can be moved in a controlled predictable way under the control of motors and/or manual handles. Up until now, the equipment manufacturers and the radiographers/clinicians have adopted the view that rigidly fixing the imaging and invasive equipment in position so that patient movement will not cause movement of the equipment is the only way to obtain accurate images and ultimately accurate positioning for equipment during invasive procedures such as biopsy. The compression paddle and the support/image receptor for the breast, the biopsy equipment as well as the radiation emitter are in a fixed position when the breast is compressed for imaging and biopsy procedure. The relative positions of compressed breast and imaging equipment are fixed to ensure that the data taken from the images for measuring the three dimensional spatial location and volume of a lesion is accurate data. This data is essential for spatially locating the invasive equipment relative to the compressed breast by using the same three dimensional coordinate geometry system for imaging and positioning of the biopsy equipment. Otherwise, it would be very difficult or impossible to accurately locate the equipment relative to the lesion. The way in which the two prior art systems remove any uncontrollable movement of these components to avoid positional inaccuracies creeping in during the procedure is by mounting them on heavy pedestals or bases which are fixed onto or into the floor either directly or through a mammography unit.

Therefore, with both prior art systems, in the event that the patient moves all or part of their body through discomfort or involuntary body movements like sneezing or coughing, the entire strain caused by the movement is absorbed by the patient's breast tissue and skin. The reason is because the position of the compression paddle and image receptor support is fixed relative to the breast for imaging and subsequent positioning of biopsy equipment. This patient movement whilst their breast is clamped can cause significant discomfort and pain/injury to the patient.

Furthermore, in the event that the stereotactic and/or scout images have been taken just before the patient moves, if the position of the breast or lesion has moved or slipped relative to the compression paddle from their original position as a result of the strain applied to the breast during the movement, then the imaging information recorded on the stereotactic images and the scout image is no longer accurate. This is because the position of the lesion has moved relative to the already calculated position using the imaging equipment and computational analysis. This means that the relative positions of the components of the mammographic device must be recalculated and further images must be taken which increases the dose of radiation as well as extending the duration of the overall procedure. The patient may also have to be repositioned relative to the table and/or compression paddle and image receptor support to ensure the lesion or suspicious tissue is still within the field of view of the imaging equipment.

Other problems exist with the prior art in that the patient must be brought into line with the system which can be difficult for older or less mobile patients. There is also a known problem with the prior art where lesions close to the axilla are difficult to image because of the sheer size and rigidity of the equipment.

In relation to the upright add on stereotactic equipment, the patient also has a clear view of procedure and many patients find this unsettling leading occasionally to a vasovagal response. In this upright add on equipment, the x-ray emitter is generally mounted on a C-arm which is itself a significant size and the movement required of the x-ray emitter causes a significant disturbance to the patient who is proximal to this C-arm. This arrangement also restricts the possible positions the patient can rest their arms during the procedure which can be lengthy, especially if it has to be recommenced as a result of breast/lesion movement.

A further drawback brought about by the sheer size of most of the stereotactic systems is their requirement for permanent installation in a dedicated room and significant planning for utilisation of floor space around the equipment itself. This consequently requires all of the patients to be brought to the installed equipment. For a device that is used periodically this is an undesirable use of scarce resources for medical facilities.

Although the problems above have been mainly described in relation to existing stereotactic breast biopsy equipment, similar problems of patient discomfort, re-imaging due to patient movement and the problem of having to bring the patient to the mammography equipment also exists with screening mammography equipment. This is due to the lack of mobility of existing mammography equipment as well as permanent mounting techniques for this equipment in existing medical facilities.

It is an object of the present invention to obviate or mitigate the problems outlined above.

Accordingly, the present invention provides a mammographic device comprising a support means for carrying a mammographic head movably mounted on the support means, the mammographic head having a breast clamping means, wherein the mammographic head is movably mounted on the support means so as to be movable in response to forces transmittable through the breast clamping means via a patient's clamped breast as a result of the patient's body movements.

In one embodiment, the mammographic device is a mammographic biopsy device.

In this embodiment, the mammographic head is a mammographic biopsy head.

Ideally, the mammographic head is movable in response to a force less than the force required to slidably move a portion of the clamped breast relative to the breast clamping means.

Preferably, the mammographic head is movable in a vertical and/or horizontal direction with the application of a force less than 75 Newtons.

Ideally, the mammographic head is movable in a vertical and/or horizontal direction with the application of a force less than 50 Newtons.

Preferably, the mammographic head is movable in a vertical and/or horizontal direction with the application of a force less than 35 Newtons.

Ideally, the mammographic head is movable in a vertical and/or horizontal direction with the application of a force greater than 10 Newtons.

Preferably, the mammographic head is movable in a vertical and/or horizontal direction with the application of a force greater than 25 Newtons.

Ideally, the mammographic head is movable in a vertical and/or horizontal direction with the application of a force of approximately 30 Newtons.

The force required to move the mammographic head is transmittable through a patient's clamped breast by movement of the patient's body during the procedure.

Ideally, the support means comprises a support member carrying a laterally protruding arm.

Preferably, the laterally protruding arm carries the mammographic head.

Advantageously, this freedom of movement of the mammographic head in response to patient movement results in the mammographic head being capable of tracking the natural movement of the patient's body and compressed breast to naturally accommodate movement of the patient's body during the procedure. This means that even if the patient's body and the patient's breast move during the procedure then the mammography head moves in the same way so there is no requirement to restart the imaging process during the procedure as a result of this patient body movement.

This ensures that the patient is exposed to less radiation and the overall procedure takes less time. The movement of the mammographic head also results in there being no unnecessary strain applied to the breast tissue or skin brought about by the transmission of forces through the breast because of the patient's body moving relative to the compressed breast. This increases the patient comfort thereby reducing patient stress levels during an already physically and emotionally challenging experience.

Ideally, the mammographic head comprising a radiation emitter and radiation receiver, breast clamping means comprising a breast support means for engaging at least a portion of a breast and compression means for compressing at least a portion of the breast between the support means and the compression means.

Preferably, the compression means is movably mounted between the radiation emitter and radiation receiver.

Ideally, the breast support means houses the radiation receiver.

Preferably, the surface of the breast compression paddle and/or the surface of the breast support means engageable with the patient's breast is provided with means for increasing friction there between.

Ideally, a cover is provided for the breast compression paddle and/or the breast support means.

Preferably, the cover is manufactured from a flexible non-slip material.

Advantageously, this increases the friction forces between the breast and the cover.

Ideally, the cover or the surface material for the breast compression paddle and/or the breast support means is disposable.

Preferably, the mammographic head comprises a biopsy means movably mounted on the head between the radiation emitter and the compression means.

In one embodiment, the radiation emitter comprises a single radiation source.

In this embodiment, the position of the single radiation source is adjustable relative to the radiation receiver. Advantageously, this allows multiple images of the breast to be taken at a range of angles.

In a second embodiment, the radiation emitter comprises more than one radiation source.

In this second embodiment, the position of the plurality of radiation sources is fixed. Furthermore, the use of fixed position radiation sources means no travel time is required for source travel after the scout image is taken reducing the overall time of the procedure. There is also greater freedom for the source to image distance SID. The risk of any collision between source equipment and any other equipment on the mammography head because of source movement is also eliminated.

Alternatively, the position of one or more of the plurality of radiation sources is adjustable relative to the radiation receiver. Advantageously, this allows multiple images of the breast to be taken at a range of angles.

Ideally, the plurality of radiation sources comprise switching means for switching a single power source between the plurality of radiation sources. Advantageously, a single power source can supply the predetermined radiation source with power when the specific radiation source is required to image the breast. This reduces the electrical cabling, conduit and space requirements and complexity of design.

Preferably, the switching means is a relay and/or electronic switching means. Switching allows one radiation source to cool down while another is in operation.

Ideally, the radiation source is of the type having a fixed as opposed to rotating anode.

Preferably, the radiation source is a carbon nanotube x-ray emitter.

Ideally, the position of the one or more fixed radiation source and the one or more movable sources is determined so that the source to image receptor distance SID is constant for the fixed sources and through the range of motion of the one or more movable radiation sources. This SID distance is swept out by a radius centred about the location of the image receiver.

Ideally, the support means comprises an upright post having an upright axis.

Preferably, the laterally protruding arm is freely rotatably movable about the support means and/or vertically adjustable about the support means.

Preferably, the laterally protruding arm is freely rotatably movable about the upright axis and/or vertically adjustable about the upright axis.

Ideally, the laterally protruding arm is pivotally movable about the support means and/or vertically adjustable about the support means.

Preferably, the laterally protruding arm is pivotally movable about the upright axis and/or vertically adjustable about the upright axis. Advantageously, the pivotal mounting of the laterally protruding arm on the support means/upright post allows the mammographic head to rotate 360 degrees around the support means/upright post.

Preferably, the mammographic head is freely rotatably movable about the support means and/or vertically adjustable about the support means.

Ideally, the mammographic head is freely rotatably movable about an upright axis and/or vertically adjustable about the upright axis.

Preferably, the mammographic head is pivotally movable about the support means and/or vertically adjustable about the support means.

Preferably, the mammographic head is pivotally movable about an upright axis and vertically adjustable about the upright axis. Advantageously, the pivotal mounting of the mammographic head on the support means/upright post allows the mammographic head to rotate 360 degrees around the support means/upright post.

Ideally, the inertial force required to cause movement of the mammographic head is calibrated to be below the minimum force required to cause relative movement between a clamped breast and the breast clamping means. The minimum force required to cause movement of the breast relative to the breast clamping means would be a force capable of overcoming the combined clamping force of the breast clamping means and the friction force acting between the breast clamping means and the breast.

Ideally, the laterally protruding arm is pivotally mounted on the upright post via a collar.

Preferably, the weight of the balancing arm and the mammographic head is distributed onto the upright post by collared bearings disposed between the collar and the upright post. Advantageously, this avoids stress points on the upright post and collar due to localized force application from the weight of the balancing arm and the mammographic head.

Ideally, the laterally protruding arm is a balanced arm having balancing means.

It will of course be appreciated that the support means and/or the laterally protruding arm may be balanced in a wide range of ways comprising active mechanism having sensors and actuators, mechanical or gas based biasing means, mechanical coupling arrangements, friction, counterweights or any combination of these means/arrangements suitable for providing a balanced arm. By use of the term balanced arm we mean that the mammographic head will always be supported on the balanced arm against gravity by the various balancing means.

Ideally, the balancing means of the laterally protruding arm is provided by one or more gas struts.

Preferably, the gas struts extend diagonally along the length of the laterally protruding arm between the ends thereof.

Preferably, the gas struts are calibrated to support the weight of the balanced arm and the mammographic head at a range of predetermined positions. Advantageously, the adjustable nature of the biasing force in the gas struts or other balancing means allows variable weights of laterally protruding arm and mammographic head combinations to be accommodated. It also provides a means for adjusting the sensitivity of patient movement tracking capability of the mammographic head by adjusting the vertical movement forces required to vertically displace the mammographic head.

Ideally, a small amount of force applied to the mammographic head in an upward direction will be supported by the action of the balancing means/gas struts to reduce the upward force required to counteract the weight of the balanced arm and the mammographic head. Advantageously, this means that a small amount of upward force applied by a patient via their breast clamped in the mammographic head is readily tracked by the mammographic head to reduce the strain on the patient's breast.

Preferably, the balancing means are calibrated so that the amount of force required to be applied to the mammographic head in an upward direction to overcome the weight of the balanced arm and the mammographic head is below the minimum force required to cause relative movement between a clamped breast and the breast clamping means.

Ideally, a small amount of force applied to the mammographic head in a downward direction will easily overcome the normally operating upward action of the balancing means/gas struts. Advantageously, this means that a small amount of downward force applied by a patient via their breast clamped in the mammographic head can be readily tracked by the mammographic head to reduce the strain on the patient's breast.

Preferably, the balancing means are calibrated so that the amount of force required to be applied to the mammographic head in a downward direction to overcome the upward force of the balancing means/gas struts normally acting on the balanced arm and the mammographic head is below the minimum force required to cause relative movement between a clamped breast and the breast clamping means.

Ideally, articulation means are provided between the end of the balanced arm distal to the support means and the mammographic head.

Preferably, the articulation means comprises a rotary hinge joint.

Ideally, the rotary hinge joint comprises a mounting bracket forming part of or being coupled to the end of the balanced arm distal to the support means having a concave recess formed for receiving an arcuate swivel plate.

Preferably, the rotary hinge joint allows the mammographic head to swivel about an upright axis relative to the balanced arm. Advantageously, this allows the mammographic head to have additional degrees of rotational movement to increase the ability to position the breast clamping means in still further angular orientations relative to the patients breast for providing the clinician with greater access to difficult to reach lesions. This rotary hinge joint provides further articulation of the mammographic head so that the clinician can bring the mammographic head to the patient instead of having to line the patient up with the very limited degrees of movement provided with the two prior art stereotactic biopsy systems or screening mammography equipment. This rotary hinge joint is particularly useful for allowing a clinician to adjust the breast clamping means into position for taking a range of imaging positions comprising craniocaudal, extended craniocaudal, mediolateral oblique, mediolateral, lateromedial, lateromedial oblique, Axillary tail, superior inferior, superior inferior oblique and inferior superior with the patient in decubitus. Advantageously, the rotary hinge also allows the mammographic head to swivel about a vertical axis to allow the compression paddle to be swivelled around the patient's body trunk.

Ideally, the articulation means further comprises a turntable arrangement movably coupled to the mammographic head.

Preferably, the articulation means further comprises a turntable arrangement pivotally coupled to the mammographic head.

Ideally, the turntable arrangement comprises a turntable brake means mounted on the mammographic head.

Preferably, the brake means is fixed in position on the mammographic head.

Preferably, the turntable arrangement has a turntable coupled to the balanced arm.

Ideally, the turntable is fixed to the balanced arm.

Ideally, the mammographic head is pivotally coupled to the turntable via a turntable pivotal coupling.

Ideally, the mammographic head is continuously rotatable about the turntable pivotal coupling.

Ideally, the brake means rotates around the circumference of the turntable as the mammographic head is pivoted about the turntable pivotal coupling.

Preferably, the brake means is engageable with the turntable for preventing rotation of the mammographic head relative to the turntable.

Ideally, the brake means comprises a solenoid operated locking bolt formed for operable engagement and disengagement with angularly spaced bores formed in the circumference of the turntable. Advantageously, this allows an operator to adjust the angular orientation of the mammographic head relative to the turntable in a plane parallel to the main plane of the turntable.

Preferably, the brake means and the turntable arrangement are housed within the protective cover of the mammographic head. Advantageously, this prevents patients from seeing any unnecessary industrial components.

Advantageously, this allows the mammographic head to have additional degrees of rotational movement to increase the ability to position the breast clamping means in still further angular orientations relative to the patients breast for providing the clinician with greater access to difficult to reach lesions. This turntable arrangement is useful for allowing a clinician to adjust the breast clamping means into position for taking a range of imaging positions comprising craniocaudal, extended craniocaudal, mediolateral oblique, mediolateral, lateromedial, lateromedial oblique, Axillary tail, superior inferior, superior inferior oblique and inferior superior with the patient in decubitus. This provides further articulation of the mammographic head so that the clinician can bring the mammographic head into the correct orientation about the patient's body without having to line the patient up with the very limited degrees of movement provided with the two prior art stereotactic biopsy systems and the mammographic equipment.

Ideally, the turntable is mounted to or is integrally formed with the rotary hinge joint.

Preferably, the turntable is mounted to or is integrally formed with the arcuate swivel plate of the rotary hinge joint.

Preferably, an extendable arm arrangement is provided between the balanced arm and the mammographic head.

Ideally, an extendable arm arrangement is provided between the rotary hinge joint and the mammographic head.

Preferably, the extendable arm arrangement is provided by a plurality of telescopic members.

Alternatively, the extendable arm is provided by two arms having an elbow joint.

Ideally, one end of one of the arms is connected to the arcuate swivel plate of the rotary hinge joint coupled to or integrally formed with the balanced arm.

Preferably, the end of the other arm distal the elbow joint is designed as an arcuate swivel plate for operable engagement with a mounting bracket having a concave recess formed for receiving the arcuate swivel plate. Advantageously, the elbow joint between the two arms and the rotary hinge joints on each end of the two arms distal the elbow joint further improves the articulation function of the mammographic head relative to the patient's body.

Ideally, the one or more rotary hinge joints have low friction bearings. Advantageously, the low friction bearings help to ensure that the force required to move the mammographic head towards and/or away from the patient's body as a result of patient body movement whilst a breast is clamped are kept well within the range of forces outlined above.

Ideally, the laterally protruding balanced arm comprises a tubular member having one end movably coupled to the support member/collar and the other end movably coupled to the rotary hinge joint.

Preferably, the laterally protruding balanced arm comprises a tubular member having one end pivotally coupled to the support member/collar and the other end movably coupled to the rotary hinge joint.

Ideally, the laterally protruding arm is a tubular member with a quadrangular cross section with each corner of one end pivotally coupled to the support member/collar and each corner of the other end pivotally coupled to the rotary hinge joint. Advantageously, this allows the laterally protruding balanced arm to move in a vertical plane about the pivotal couplings.

Ideally, the telescopic arm arrangement comprises two telescoping members and a mounting means for mounting the telescopic arrangement on the mammographic head and the rotary hinge joint. Advantageously, the telescopic arm arrangement allows an operator to move the mammographic head towards and away from the body of the patient when the base or support member is in a fixed position.

Ideally, the mammographic head has a generally C-shaped arm having a rear face on the spine of the C-shaped arm mechanically coupled to the supporting means/balanced arm outside the space enclosed by the C-shaped arm. It will of course be appreciated that other shapes of C-arm can be utilized with the invention.

Preferably, the C-shaped arm has a front face on the spine defining a guide track for receiving the compression paddle protruding from the guide track into the space enclosed by the C-shaped arm.

Ideally, the C-shaped arm has a front face on the spine defining a guide track for receiving a biopsy equipment carriage means protruding from the guide track into the space enclosed by the C-shaped arm.

Preferably, the biopsy carriage means carries a range of biopsy needle support equipment.

Ideally, a range of needles and other biopsy equipment are supportable in this equipment.

Preferably, the C-shaped arm has a head positioning means extending along at least part of one lateral edge of the C-shaped arm.

Ideally, the head positioning means is provided by a handle/rail.

Ideally, the head positioning means has means for actuating the brake means of the turntable arrangement.

Preferably, one flange of the C-shaped arm defines the breast support means.

Ideally, the breast support means has manually adjustable extension means for increasing the surface area of the breast support means. This allows the breast support means to accommodate a greater range of breast shapes and sizes.

Preferably, the breast support means houses the radiation receiver.

Ideally, the radiation receiver is provided by a flat panel detector.

Preferably, the radiation receiver is electrically/electronically coupled to a computer for calculating the location of the lesion using stereotactic, stereotactic and scout, or tomosynthesis.

Ideally, the position of the radiation receiver is adjustable within the breast support means.

Preferably, the radiation receiver is mounted on powered motive means.

Ideally, the powered motive means of the radiation emitter are coupled to the control means.

Preferably, the position of the radiation receiver is adjustable in a predetermined way in response to positional adjustment of the radiation emitter. Advantageously, this allows a range of imaging techniques to be used where these images are useful to provide different views of the breast tissue.

Preferably, the breast support means is dimensioned to allow the entire breast support means to rest under the patient's breast engaging the axilla.

Ideally, the other flange of the C-shaped arm carries a screen protruding from the flange and extending along a part of the space defined between the two flanges. Advantageously, the screen blocks a patient's view of the radiation emitter and the biopsy equipment. This occurs in all imaging positions.

Ideally, the radiation emitter is movably mounted on guide means which is coupled to the mammographic head.

Ideally, the guide means is designed so that the source to image receptor distance is constant through the range of motion of the radiation source. Ideally, the guide means is designed so that the radiation emitter moves about a circumference of a circle swept out by a radius centred about the location of the image receiver.

Preferably, the radiation emitter is movably mounted on a guide track which is coupled to the C-shaped arm.

Ideally, the guide track follows the circumference of a circle swept out by a radius centred about the location of the image receiver.

Ideally, the radiation emitter is movably mounted on a guide track which is coupled to the inside surface of the screen.

Preferably, the guide track is arcuate.

Preferably, the radiation emitter is mounted on a carriage means movable along the guide track by powered motive means.

Ideally, the powered motive means are operaply coupled to a computer for controlling the angular displacement of the radiation emitter along the guide track. Advantageously, in this arrangement, there is no requirement for movement of any section of the C-arm in order to take a number of angularly displaced images of the breast as the radiation emitter is simply moved along the guide track. The screen blocks the patient's view of this movement and reduces the anxiety levels for the patient.

Preferably, the emitter is an x-ray emitter with a target and filtration combination which will result in an x-ray beam quality suitable for mammographic imaging. Any x-ray emitter used must meet specific regulatory requirements for this procedure.

Ideally, the mammographic device is portable.

Accordingly, the present invention provides an upright mammographic device comprising a support means for carrying a mammographic head movably mounted on the support means, the mammographic head having an arm movably coupled to the support means for carrying a radiation emitter, the radiation emitter being mounted on guide means coupled to the arm, the radiation emitter being movable via the guide means when the arm is fixed in position relative to a patient and the support means and when the arm is movable relative to the support means.

Advantageously, this mobility of the radiation source when the arm is fixed relative to the support means and patient allows the clinician/radiographer to initially move the arm into the appropriate position relative to the patient's body for the required imaging orientation. Then, when the additional images have to be taken, the parts of the arm which are in contact with the patient are not required to be moved because the radiation source is movable without requiring any movement of the remainder of the structure of the arm. This prevents the patient from seeing, feeling and hearing large parts of the equipment undergoing movements during the imaging procedure. In the prior art the section of the C-arm housing the radiation emitter of the mammographic head rotates for imaging because the radiation source is fixed in position on one end of the C-arm. This causes significant distress to the patient and risks causing patient movement due to the moving section of the C-arm. The mobility of the radiation source of the present invention when the arm is fixed relative to the patient and support means removes this patient discomfort and prevents the patient from feeling, seeing or hearing movement of major components of the arm during the imaging phase.

Preferably, the arm carries a breast clamping means and a radiation receiver.

Ideally, the arm is a generally C-shaped arm.

Ideally, the radiation emitter is movably mounted on the guide means which is coupled to the arm of the mammographic head.

Preferably, the radiation emitter is movably mounted on guide means being a guide track which is coupled to the arm.

Ideally, the radiation emitter is movably mounted along a guide track which is coupled to the inside surface of the screen.

Ideally, the guide means is designed so that the source to image receptor distance is constant through the range of motion of the radiation source. Ideally, the guide means is designed so that the radiation emitter moves about a circumference of a circle swept out by a radius centred about the location of the image receiver.

Preferably, the guide track is arcuate.

Preferably, the radiation emitter is mounted on a carriage means movable along the guide means by powered motive means.

Ideally, the mammographic device is portable.

A mammographic device comprising a support means for carrying a mammographic head mounted on the support means, the mammographic head having a radiation emitter comprising more than one radiation source.

Ideally, the mammographic head is movably mounted on the support means.

Preferably, the mammographic device is an upright mammographic device.

Ideally, the plurality of radiation sources comprise switching means for switching a single power source between the plurality of radiation sources. Advantageously, a single power source can supply the predetermined radiation source with power when the specific radiation source is required to image the breast. This reduces the electrical cabling, conduit and space requirements and complexity of design.

Preferably, the switching means is a relay and/or electronic switching means. Switching allows one radiation source to cool down while another is in operation.

Ideally, the radiation source is of the type having a fixed as opposed to rotating anode.

Preferably, the radiation source is a carbon nanotube x-ray emitter.

Ideally, the mammographic device further comprises a radiation receiver and breast clamping means, the breast clamping means comprising a breast support means for engaging at least a portion of a breast and compression means for compressing at least a portion of the breast between the support means and the compression means.

Preferably, the compression means is movably mounted between the radiation emitter and radiation receiver.

Ideally, the breast support means houses the radiation receiver.

Preferably, the surface of the breast compression paddle and/or the surface of the breast support means engageable with the patient's breast is provided with means for increasing friction there between.

Ideally, a cover is provided for the breast compression paddle and/or the breast support means.

Preferably, the cover is manufactured from a flexible non-slip material. Advantageously, this increases the friction forces between the breast and the cover.

Ideally, the cover or the surface material for the breast compression paddle and/or the breast support means is disposable.

Preferably, the mammographic head comprises a biopsy means movably mounted on the head between the radiation emitter and the compression means.

In this second embodiment, the position of the plurality of radiation sources is fixed.

Furthermore, the use of fixed position radiation sources means no travel time is required for radiation source travel after the scout image is taken reducing the overall time of the procedure. There is also greater freedom for the source to image distance SID. The risk of any collision between source equipment and any other equipment on the mammography head because of source movement is also eliminated.

Alternatively, the position of the plurality of radiation sources is adjustable relative to the radiation receiver. Advantageously, this allows multiple images of the breast to be taken at a range of angles.

Ideally, the position of the more than one radiation sources is determined so that the source to image receptor distance SID is constant for the sources. This SID distance is swept out by a radius centred about the location of the image receiver.

In one embodiment, the mammographic device is a mammographic biopsy device.

In this embodiment, the mammographic head is a mammographic biopsy head.

Ideally, the mammographic head is movably mounted on the support means so as to be movable in response to forces transmittable through the breast clamping means via a patient's clamped breast as a result of the patient's body movements.

Ideally, the mammographic head is movable in response to a force less than the force required to slidably move a portion of the clamped breast relative to the breast clamping means.

Ideally, the support means comprises a support member carrying a laterally protruding arm.

Preferably, the mammographic device is portable.

It will of course be appreciated that all of the technical features listed as ideal or preferable options from and including the statement on page 7 namely ideally, the support means comprises an upright post having an upright axis to and including the statement on page 15 namely preferably, the emitter is an x-ray emitter with a target and filtration combination which will result in an x-ray beam quality suitable for mammographic imaging in relation to the patient tracking mammography device are also appendable to the main statement of invention in relation to this multi-source emitter mammography device.

The invention will now be described with reference to the accompanying drawings which show by way of example only a mammographic device and a mammographic biopsy device in accordance with the invention. In the drawing:—

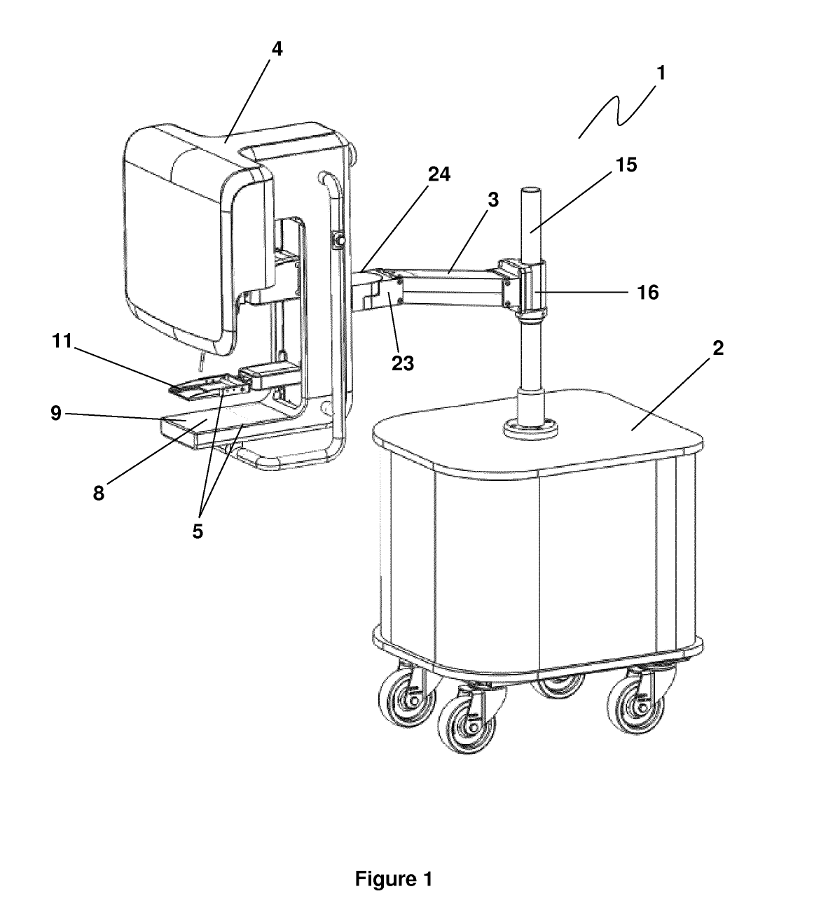

FIG. 1 is a perspective view of a mammographic biopsy device;

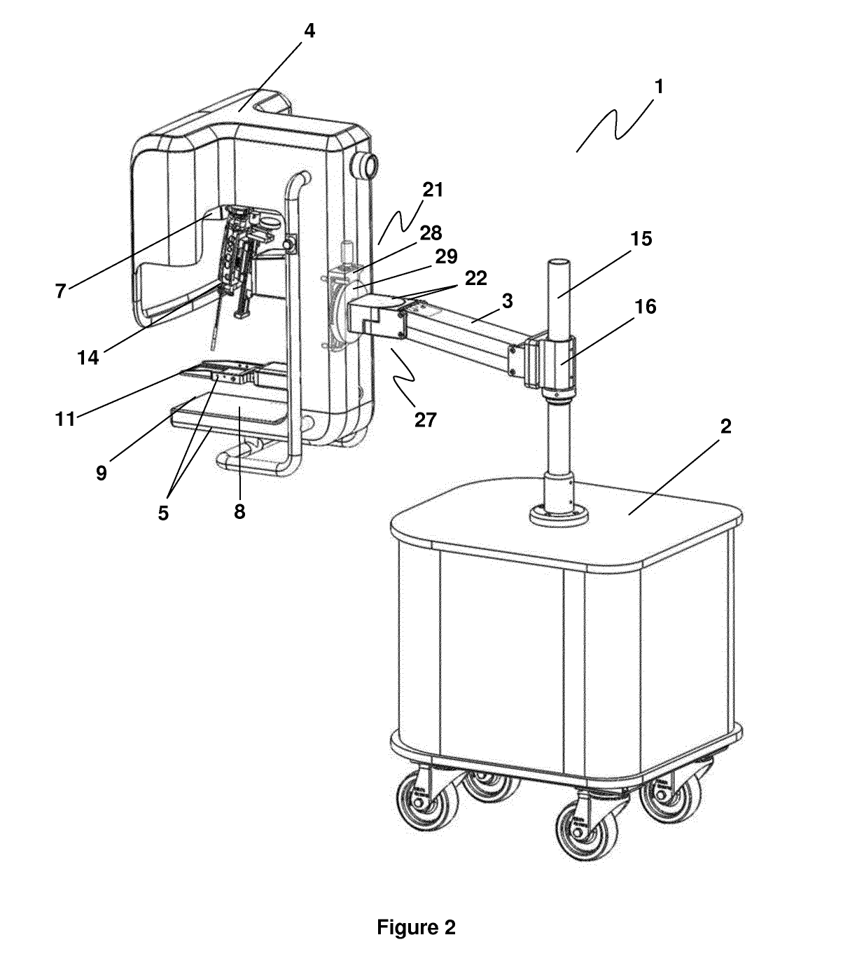

FIG. 2 is a second perspective view of a mammographic biopsy device of FIG. 1;

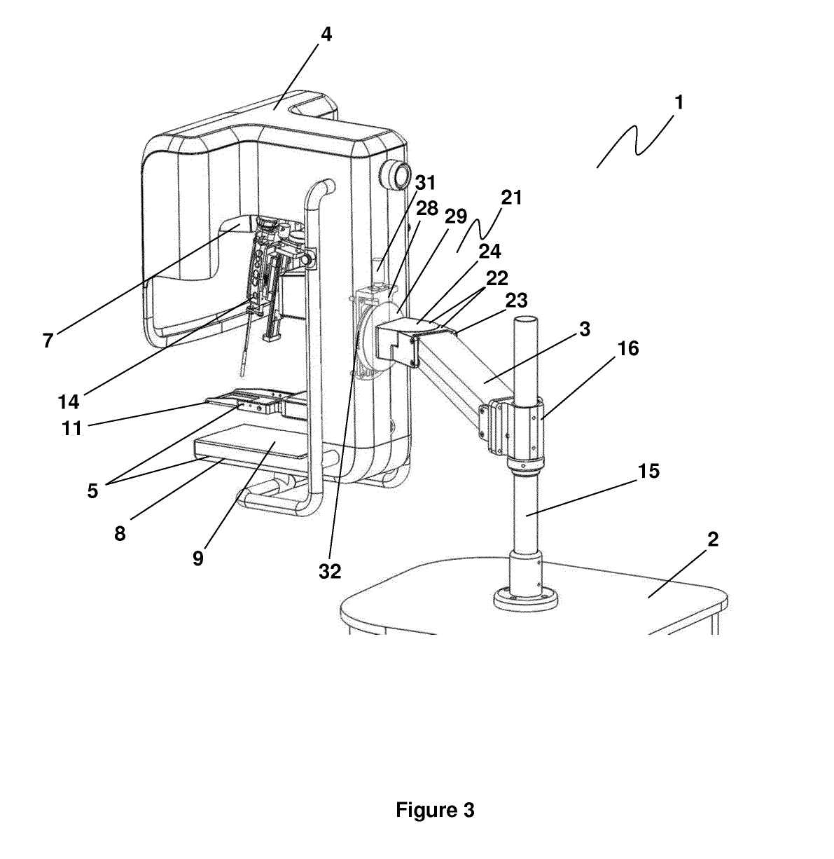

FIG. 3 is a detail perspective view of a mammographic biopsy head;

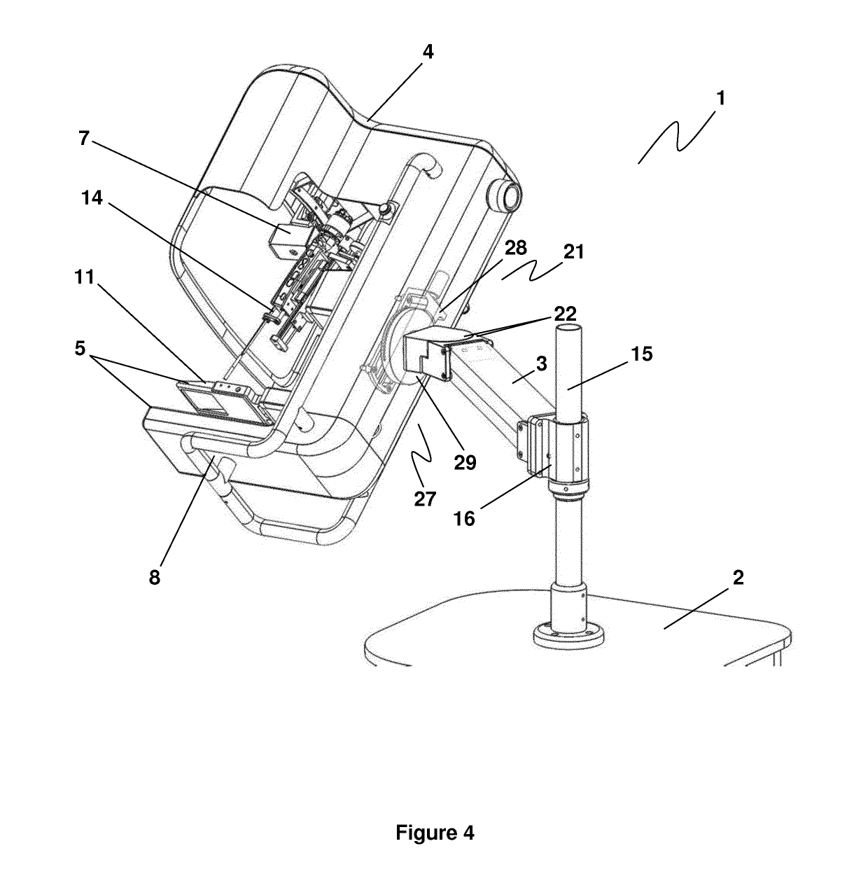

FIG. 4 is a detail perspective view of a mammographic biopsy head at a second angular orientation;

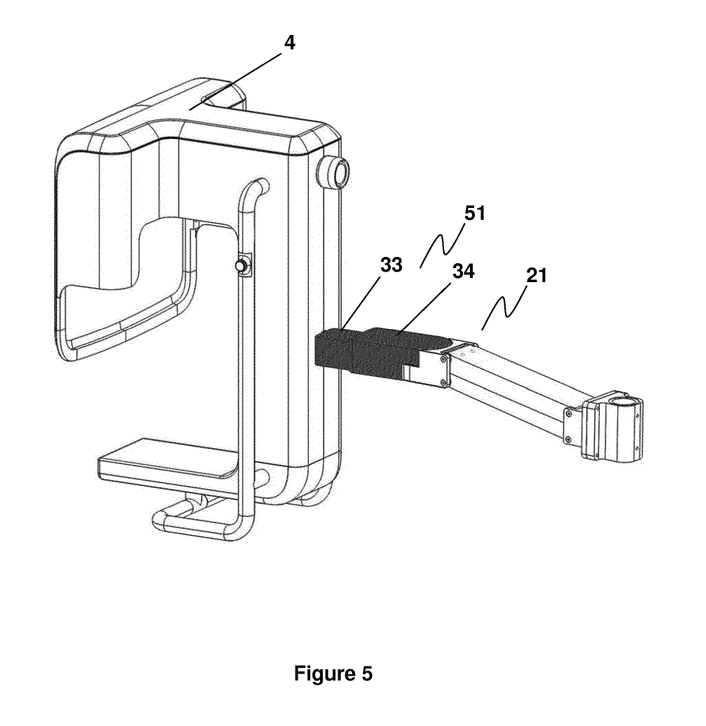

FIG. 5 is a detail perspective view of a further embodiment of mammographic head;

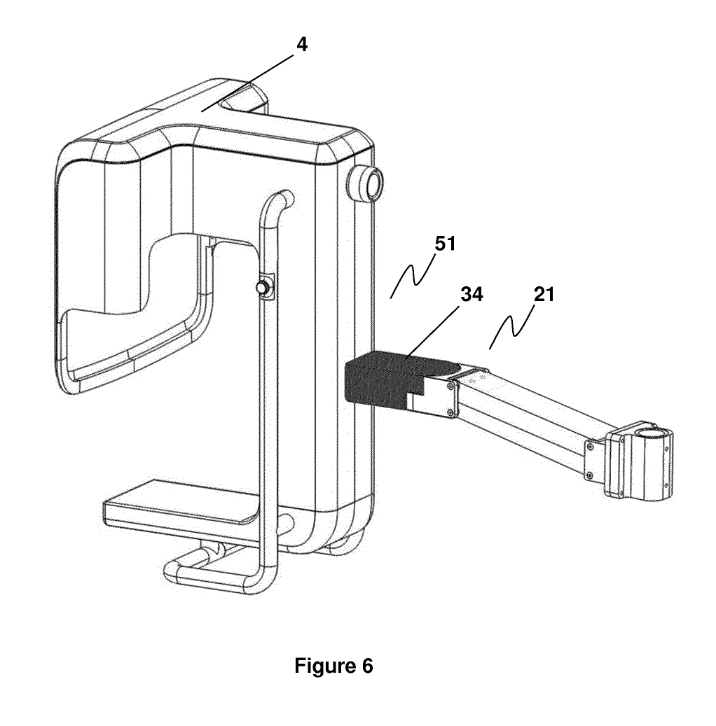

FIG. 6 is a detail perspective view of the mammographic head of FIG. 5 in a second position;

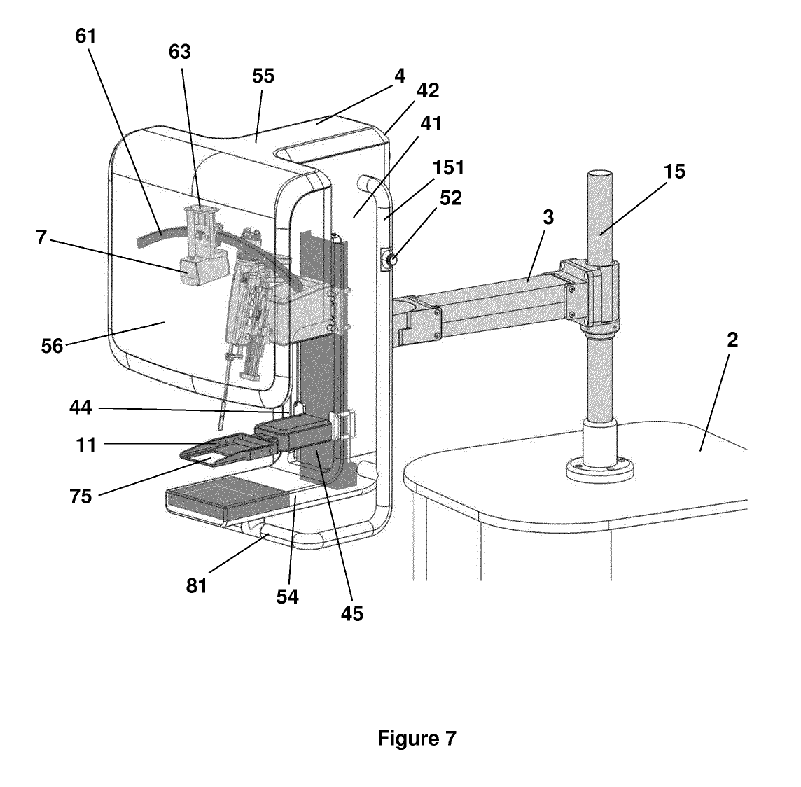

FIG. 7 is a further detail perspective view of the mammographic biopsy device;

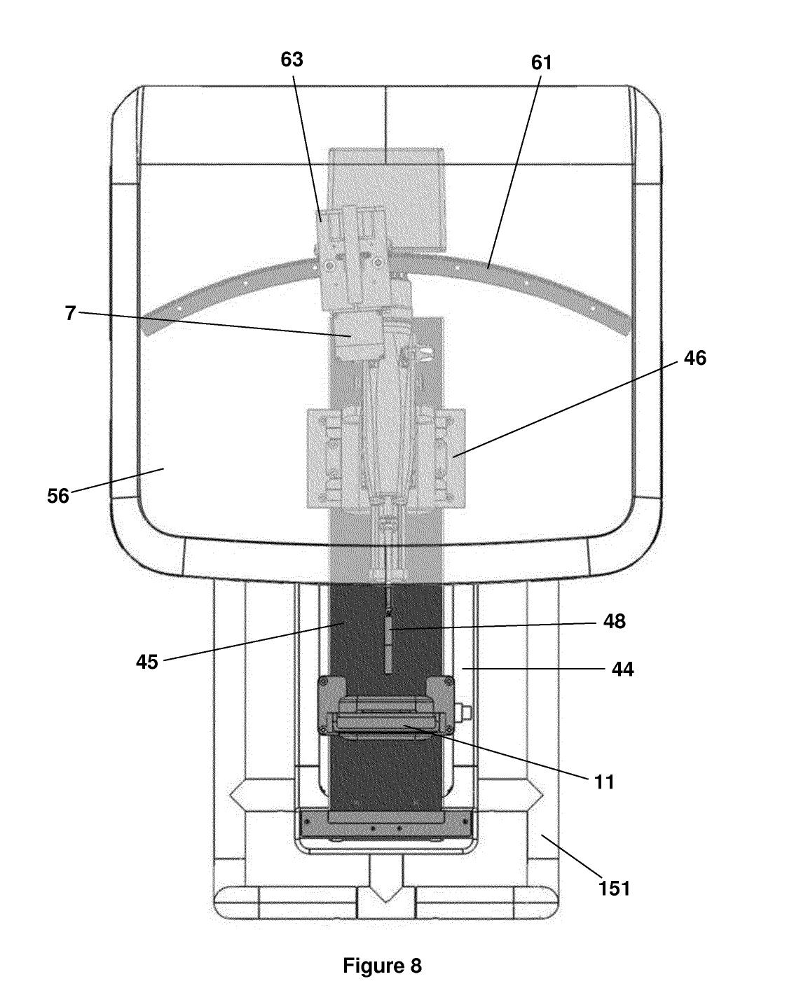

FIG. 8 is a front elevation view of the mammographic biopsy head of FIG. 7; and

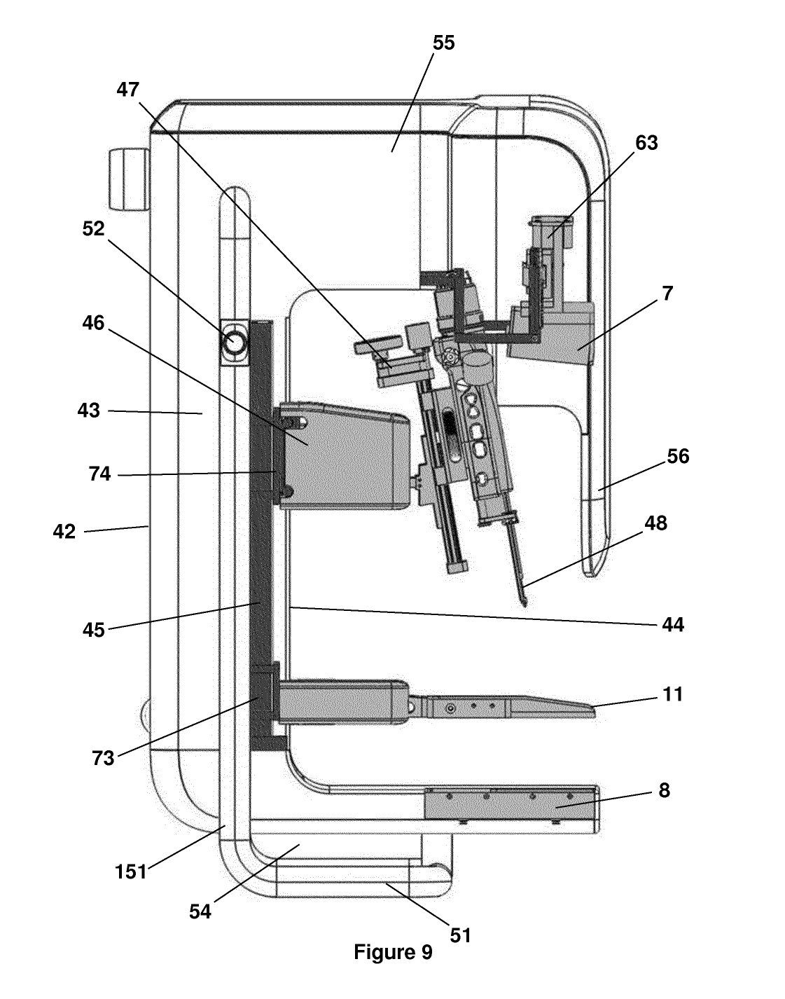

FIG. 9 is a side view of the mammographic biopsy head of FIGS. 7 and 8;

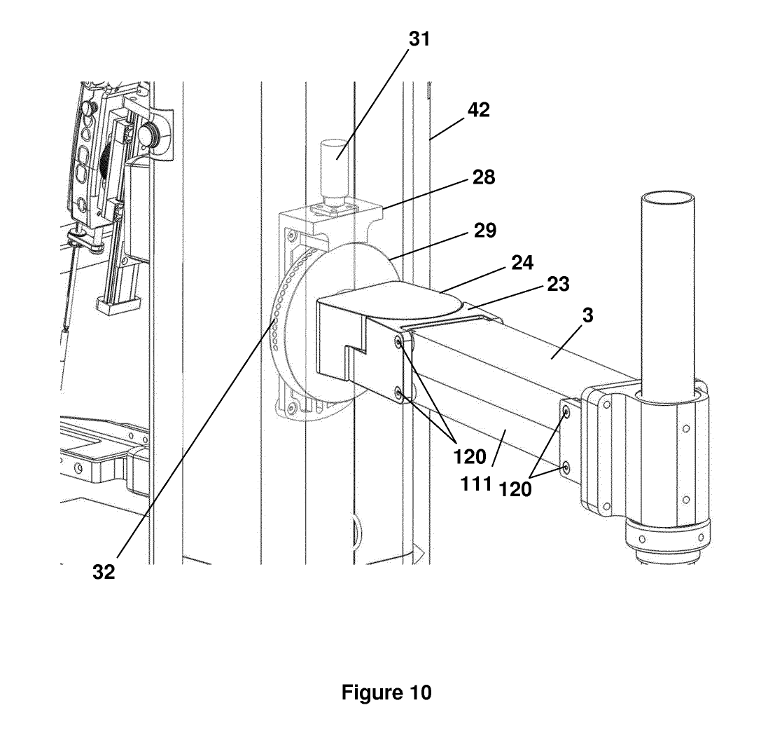

FIG. 10 is a detail view of the rotary hinge joint and turntable arrangement;

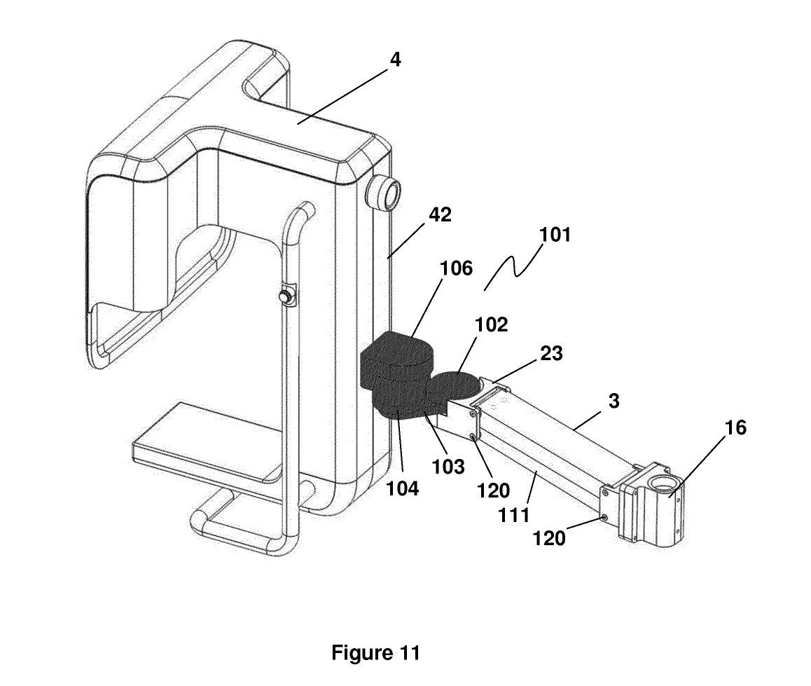

FIG. 11 is a perspective view of an alternative arrangement of extendable arm for a mammographic head;

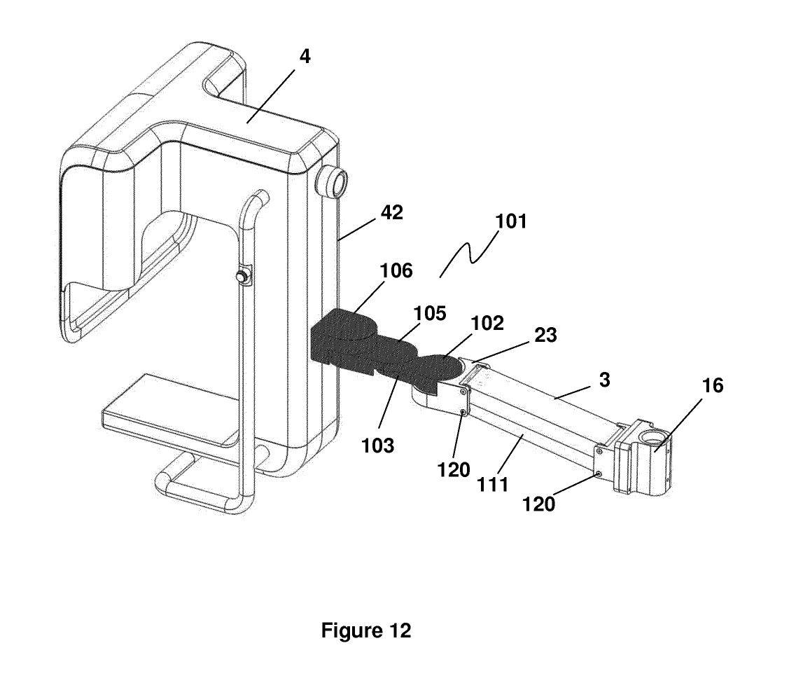

FIG. 12 is a second perspective view of an alternative arrangement of extendable arm for the mammographic head of FIG. 11 in a second position;

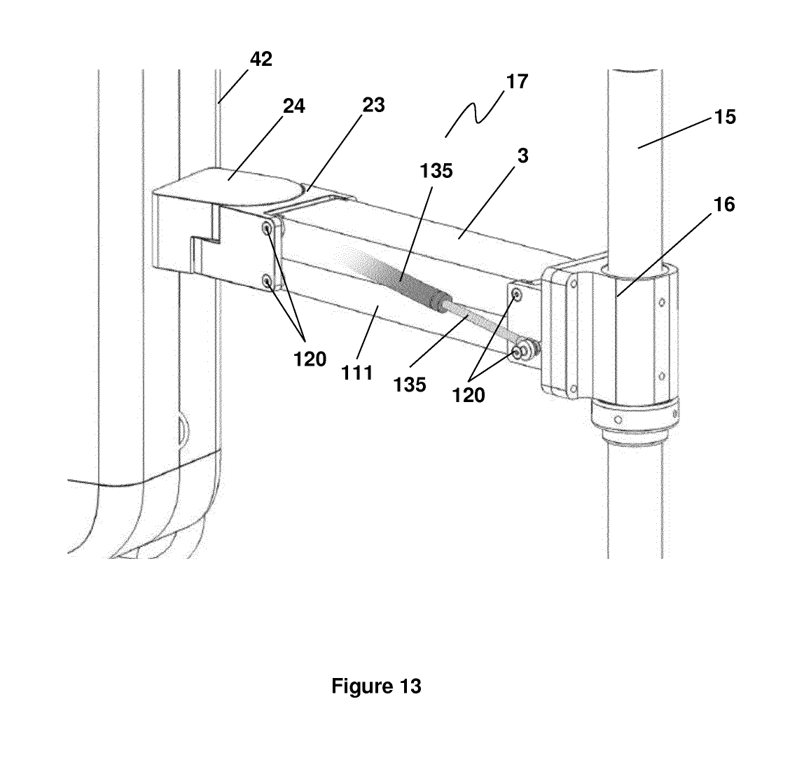

FIG. 13 is a detail perspective view of the balancing arrangement;

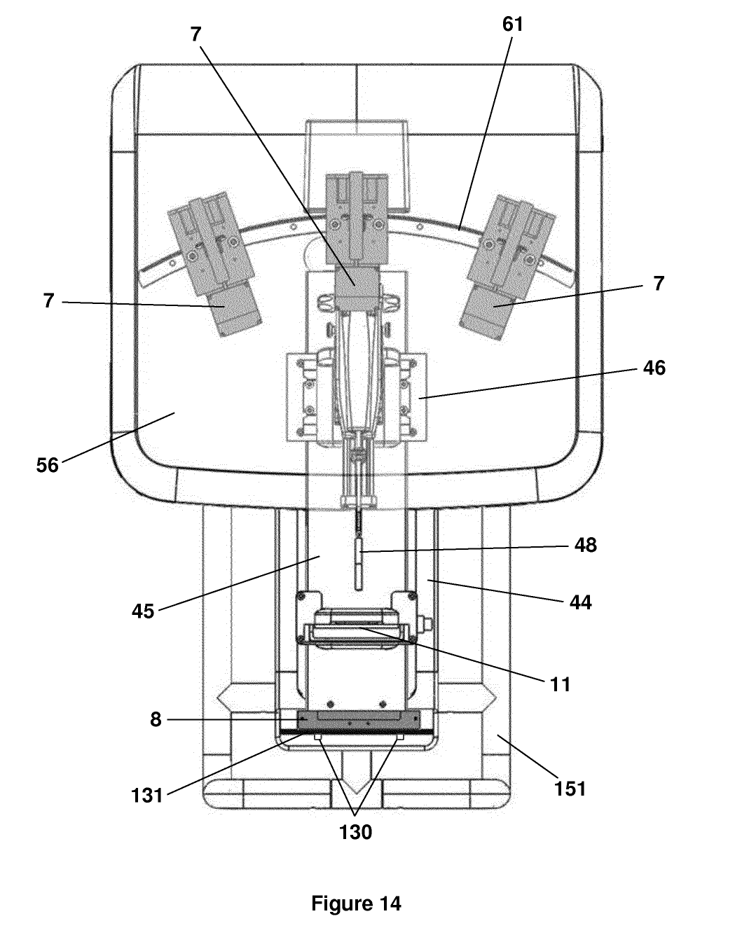

FIG. 14 is a front elevation view of the mammographic biopsy head with three radiation sources and a movable radiation receiver;

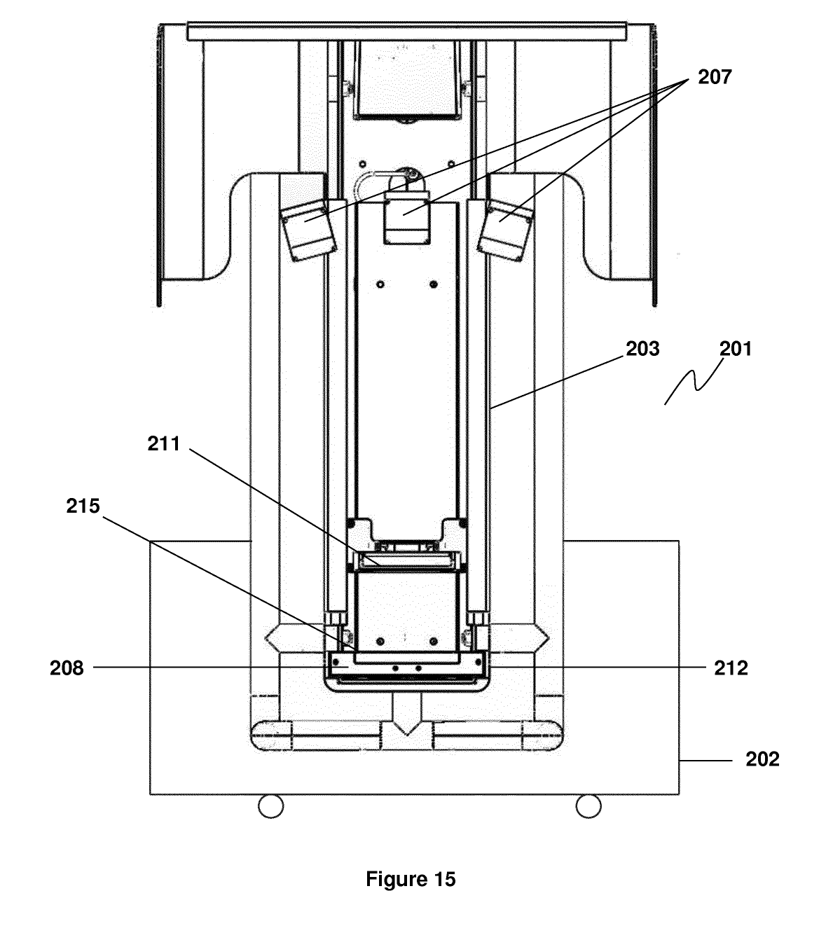

FIG. 15 is a front elevation view of a further embodiment of the invention having multiple source radiation emitters;

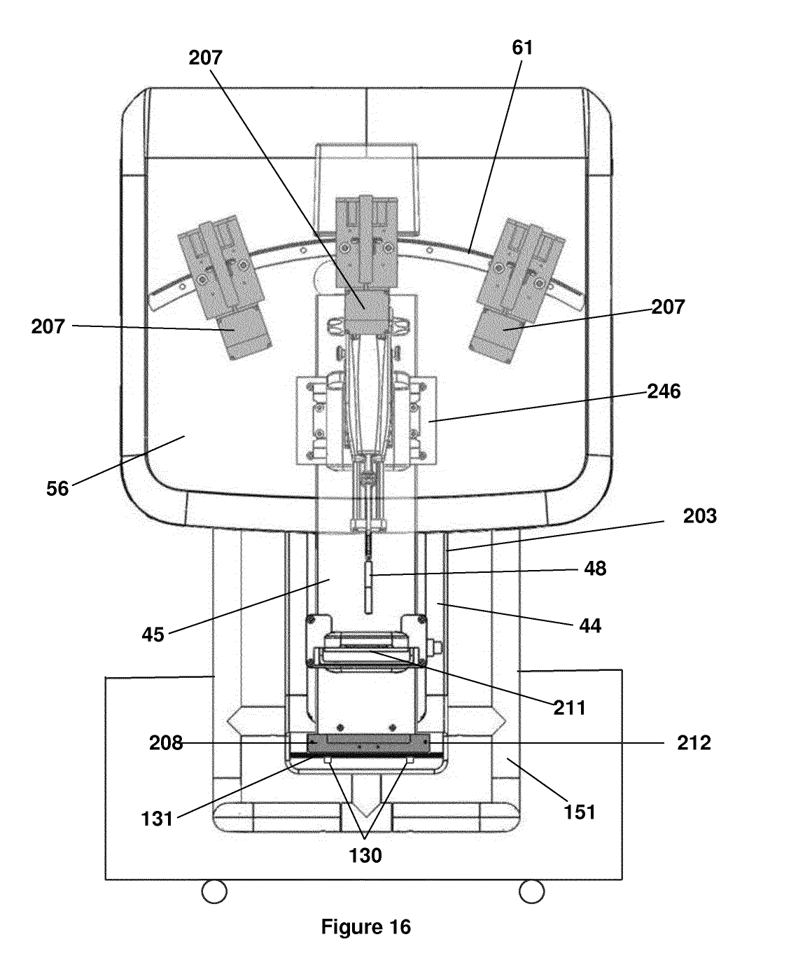

FIG. 16 is a front elevation view of a further embodiment of the invention of FIG. 15 having multiple source radiation emitters and biopsy equipment; and

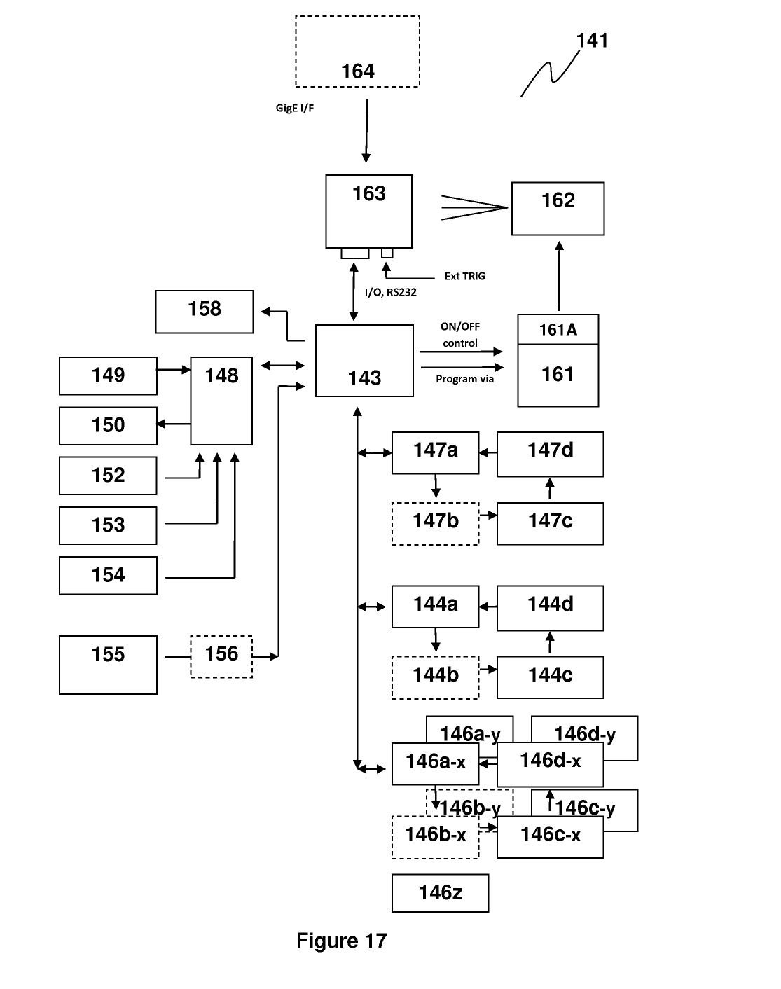

FIG. 17 is a schematic view of the overall control system of the present invention.

Referring to the drawings and initially to FIGS. 1 to 4, there is shown a mammographic biopsy device indicated generally by the reference numeral 1.

The mammographic biopsy device 1 has a support arrangement having a support member 2 provided in the embodiment shown as a trolley 2. It will of course be appreciated that a support arrangement could be suspended from the ceiling/wall of a room or a mobile van as opposed to being mounted on a trolley. The support arrangement also has a laterally protruding arm 3. The laterally protruding arm 3 supports a mammographic biopsy head 4 movably mounted on the laterally protruding arm 3. Although some of the drawings are described in relation to mammographic biopsy heads, it will of course be appreciated that the fundamental principles of the invention will work equally well with a mammographic head for screening. The mammographic biopsy head 4 has a breast clamping arrangement 5. The mammographic biopsy head 4 is movably mounted on the support arrangement 2 so as to be freely movable in response to a force less than the force required to slidably move a portion of a clamped breast (not shown) relative to the breast clamping arrangement 5.

The forces required to move the mammographic biopsy head 4 are transmittable by a patient's clamped breast through movement of the patient's body during the procedure.

Advantageously, this freedom of movement of the mammographic biopsy head 4 in response to patient movement results in the mammographic biopsy head 4 being capable of tracking the natural movement of the patient's body and compressed breast to naturally accommodate movement of the patient's body during the procedure. This means that even if the patient's body and the patient's breast move during the procedure then the mammography biopsy head 4 moves in exactly the same way so there is no requirement to recalculate relative positions of components of the equipment 1 and restart the imaging process during the procedure as a result of this patient body movement. This ensures that the patient is exposed to less radiation and the overall procedure takes less time. The movement of the mammographic biopsy head 4 also results in there being limited strain applied to the breast tissue or skin brought about by the transmission of forces through the breast because of the patient's body moving relative to the compressed breast. This increases the patient comfort thereby reducing patient stress levels during an already physically and emotionally challenging experience. The mammographic head is movable in a vertical direction with the application of a force in a range of 10 to 75 Newtons with the range being limited to 30 Newtons or less where the inclined or declined angle of the balanced arm relative to the horizontal is ±45°.

The mammographic biopsy head 4 has a radiation emitter 7 and radiation receiver 8. The breast clamping arrangement 5 has a breast support member 9 for engaging at least a portion of a breast and breast compression paddle 11 for compressing at least a portion of the breast between the breast support member 9 and the compression paddle 11. The compression paddle 11 is movably mounted on the head 4 between the radiation emitter 7 and radiation receiver 8, see FIG. 14. The breast support member 9 of the present embodiment houses the radiation receiver 8. The mammographic biopsy head 4 has biopsy equipment 14 movably mounted on the head 4 between the radiation emitter 7 and the compression paddle 11. The support arrangement 2 has an upright post 15 defining an upright axis. The laterally protruding arm 3 is freely rotatably movable about the upright post 15 and vertically adjustable along the length of the upright post 15. The laterally protruding arm 3 is pivotally movable about the upright post 15 and vertically adjustable along the length of the upright post 15. Advantageously, the pivotal mounting of the laterally protruding arm 3 on the upright post 15 allows the mammographic biopsy head 4 to rotate 360 degrees and beyond around the upright post 15. The mammographic biopsy head 4 is freely rotatably movable about the upright post 15 and vertically adjustable along the length of the upright post 15. The mammographic biopsy head 4 is pivotally movable about the upright post 15 and vertically adjustable along the length of the upright post 15. Advantageously, the pivotal mounting of the laterally protruding arm 3 on the upright post 15 allows the mammographic biopsy head 4 to rotate 360 degrees and beyond around the upright post 15.

The inertial force required to cause movement of the mammographic biopsy head 4 is calibrated to be below the minimum force required to cause movement of a clamped breast relative to the breast clamping arrangement 5.

The laterally protruding arm 3 is pivotally mounted on the upright post 15 via a collar 16. The balancing arrangement of the laterally protruding arm 3 is provided by two gas struts 135, one of which is shown in FIG. 13 although any number of struts can be incorporated to suit the forces. The gas struts 135 extend diagonally along the length of the laterally protruding arm 3 between the ends thereof. The gas struts are calibrated to support the weight of the balanced arm and the mammographic head at a range of predetermined positions. Advantageously, the adjustable nature of the biasing force in the gas struts or other balancing arrangement where used allows variable weights of laterally protruding arm 3 and mammographic head 4 combinations to be accommodated. It also provides a means for adjusting the sensitivity of patient movement tracking capability of the mammographic head 4. This can work by adjusting the movement forces required to displace the mammographic head.

The gas struts are calibrated to support the weight of the balanced arm 3 and the mammographic biopsy head 4 at a range of predetermined positions. A small amount of force applied to the mammographic biopsy head 4 in an upward direction will be supported by the action of the gas struts to reduce the upward force required to counteract the weight of the balanced arm 3 and the mammographic biopsy head 4. Advantageously, this means that a small amount of upward force applied by a patient via their breast clamped in the mammographic biopsy head 4 can be tracked by the mammographic biopsy head 4 to reduce the strain on the patient's breast.

The balancing arrangement 17 is calibrated so that the amount of force required to be applied to the mammographic biopsy head 4 in an upward direction to overcome the weight of the balanced arm 3 and the mammographic biopsy head 4 is below the minimum force required to cause movement between a clamped breast and the breast clamping arrangement 5. A small amount of force applied to the mammographic biopsy head 4 in a downward direction will easily overcome the normally operating upward action of the gas struts. Advantageously, this means that a small amount of downward force applied by a patient via their breast clamped in the mammographic biopsy head 4 can be readily tracked by the mammographic biopsy head 4 to reduce the strain on the patient's breast. The balancing arrangement 17 is calibrated so that the amount of force required to be applied to the mammographic biopsy head 4 in a downward direction to overcome the upward force of the balancing arrangement 17 normally acting on the balanced arm 3 and the mammographic biopsy head 4 is below the minimum force required to cause movement of a clamped breast relative to the breast clamping arrangement 5.

An articulation arrangement indicated generally by the reference numeral 21 is provided between the end of the balanced arm 3 distal to the support member 2 and the mammographic biopsy head 4. The articulation arrangement 21 has a rotary hinge joint 22. The rotary hinge joint 22 has a mounting bracket 23 forming part of or being coupled to the end of the balanced arm 3 distal to the support member 2 having a concave recess formed for receiving an arcuate swivel plate 24. The rotary hinge joint 22 allows the mammographic biopsy head 4 to swivel about an upright axis relative to the balanced arm 3. Advantageously, this allows the mammographic biopsy head 4 to have additional degrees of rotational movement to increase the ability to position the breast clamping arrangement 5 in still further angular orientations relative to the patients breast for providing the clinician with greater access to difficult to reach lesions. This rotary hinge joint 22 provides further articulation of the mammographic biopsy head 4 so that the clinician can bring the mammographic biopsy head 4 to the patient instead of having to line the patient up with the very limit degrees of movement provided with the two prior art stereotactic biopsy systems or screening mammographic equipment. This rotary hinge joint 22 is particularly useful for allowing a clinician to adjust the breast clamping arrangement 5 into position for taking a range of images comprising mediolateral, mediolateral oblique, lateromedial, lateromedial oblique, Axillary Tail and superior inferior oblique images.

The articulation arrangement 21 also has a turntable arrangement indicated generally by the reference numeral 27 movably coupled between the mammographic biopsy head 4 and the balanced arm 3. The articulation arrangement 21 has the turntable arrangement 27 pivotally coupled to the mammographic biopsy head 4. The turntable arrangement 27 has turntable brake 28 fixed on the mammographic biopsy head 4. The turntable arrangement 27 has a turntable 29 fixed to the rotary hinge joint 22/106 or extendable arm 51 see FIGS. 5 and 6 about a centre point of the turntable 29. The turntable brake 28 and/or mammographic biopsy head is pivotally coupled to the turntable 29. The mammographic biopsy head 4 is continuously rotatable about its pivotal coupling to the turntable 29. The brake 28 rotates around the circumference of the turntable 29 as the mammographic head is rotated. The brake 28 is engageable with the turntable 29 for preventing rotation of the mammographic biopsy head 4 relative to the turntable 29 at the behest of the clinician. The brake 28 comprises a solenoid operated locking bolt 31, see FIG. 10 formed for operable engagement and disengagement with angularly spaced bores 32 formed in the circumference of the turntable 29. Advantageously, this allows an operator to adjust the angular orientation of the mammographic biopsy head 4 relative to the turntable 29 in a plane parallel to the main plane of the turntable 29. It will of course be appreciated than any type of mechanical, electro-mechanical, pneumatic, electric, electronic or hydraulic braking arrangement or any combination of these other than a solenoid and turntable/bore arrangement can be used to fix the angular orientation of the head.

Advantageously, this allows the mammographic biopsy head 4 to have additional degrees of rotational movement to increase the ability to position the breast clamping arrangement 5 in still further angular orientations relative to the patients breast for providing the clinician with greater access to difficult to reach lesions. This turntable arrangement 27 is particularly useful for allowing a clinician to adjust the breast clamping arrangement 5 into position for taking mediolateral oblique, mediolateral, lateromedial, lateromedial oblique, Axillary Tail, superior inferior oblique images and inferior superior with the patient in decubitus. This provides further articulation of the mammographic head 4 so that the clinician can bring the mammographic biopsy head 4 into the correct orientation about the patient's body without having to line the patient up with the very limited degrees of movement provided with the two prior art stereotactic biopsy systems or the mammography equipment. The turntable 29 is mounted to or is integrally formed with the rotary hinge joint 22/106 or extendable arm 51. The turntable 29 is mounted to or is integrally formed with the arcuate swivel plate 24/106 of the rotary hinge joint 22 or extendable arm 51.

Referring to the drawings and now in particular to FIGS. 5 and 6, there is illustrated a mammographic biopsy head 4 having a telescopic arm arrangement indicated generally be the reference numeral 51 provided between the articulation arrangement 21 and the mammographic biopsy head 4. The telescopic arm arrangement 51 has two telescoping poles or tubes 33, 34 for mounting the telescopic arrangement 51 on the mammographic biopsy head 4 and the articulation arrangement 21. Advantageously, the telescopic arm arrangement 51 allows an operator to move the mammographic biopsy head 4 laterally towards and away from the body of the patient when the base or support member 2 is in a fixed position such as when brakes are applied to the wheels.

Referring to the drawings and more particularly to FIGS. 7 to 9, the mammographic biopsy head 4 has a generally C-shaped arm 41 having a rear face 42 on the spine 43 of the C-shaped arm 41 mechanically coupled to the balanced arm 3 outside the space enclosed by the C-shaped arm 41. The C-shaped arm 41 has a front face 44 on the spine 43 defining a guide track 45 for receiving the compression paddle 11 protruding from the guide track 45 generally orthogonally into the space enclosed by the C-shaped arm 41. The C-shaped arm 41 has the front face 44 on the spine 43 defining the guide track 45 which is also capable of supporting a biopsy equipment carriage 46 protruding from the guide track 45 into the space enclosed by the C-shaped arm 44. This carriage carries a range of biopsy needle support equipment 47 and the needle 48 is held in this equipment 47. The location of the needle 48 is adjustable in the X, Y and Z coordinates of the Cartesian coordinate system or angle and displacement in the polar coordinate system.

The C-shaped arm 41 has an L-shaped rail 151 extending along part of each lateral edge of the C-shaped arm 41. The rail 151 has a controller 52 for actuating the brake 28 of the turntable arrangement 27 preferably located on the opposing section of the rail 151 facing the arm 41. One flange 54 of the C-shaped arm 41 defines the breast support member 9. The breast support member 9 has manually adjustable extension members (not shown) to accommodate a range of breast shapes and sizes. These extension members may be telescopic shelf components easily manually slidable out from the two lateral edges and the leading edge of the breast support member 9 to prevent any breast material hanging uncomfortably over the edge of the breast support member 9 and to optimize the imaging process. An additional plate with an uninterrupted surface which sits over member 9 could also be utilized to achieve this function. The breast support member 9 houses the radiation receiver 8.

The radiation receiver 8 is provided by a flat panel detector utilizing direct or indirect detection that can achieve the sensitivity, spatial resolution and regulatory requirements for this application. The radiation receiver 8 and radiation emitter 7 are electrically/electronically coupled to a computer see FIG. 15 for controlling the operation of the emitter 7 and receiver 8 and for calculating the precise location of any lesion using stereotactic, stereotactic and scout or tomosynthesis. The breast support member 9 is dimensioned to allow the entire breast support member 9 to rest under the patient's breast engaging the axilla between the chest wall and a patient's arm. The breast support member 9 has dimensions of 350 mm length by 250 mm breadth by 60 mm depth although of course it will be appreciated that this is one preferred dimensional configuration only and that a wide range of dimensions can be employed without departing from the scope of the invention. The other flange 55 of the C-shaped arm 41 carries a screen 56 protruding from the flange 55 and extending along a part of the space defined between the two flanges 54, 55. Advantageously, the screen 56 blocks a patient's view of the radiation emitter 7 and the biopsy equipment 47.

The radiation emitter 7 is movably mounted on guide track 61 which is coupled to the mammographic biopsy head 4. The radiation emitter 7 is movably mounted on the guide track 61 which is coupled to the C-shaped arm 41 and more specifically the radiation emitter 7 is movably mounted on the guide track 61 which is coupled to the inside surface of the screen 56. The guide track 61 is arcuate. The guide track 61 is designed so that the source to image receptor distance is constant through the range of motion of the radiation source 7. The guide track 61 is designed so that the radiation emitter moves about a circumference of a circle swept out by a radius centred about the location of the image receiver 8.

The radiation emitter 7 is mounted on a carriage 63 movable along the guide track 61 by a powered motive assembly. The powered motive assembly is operaply coupled to a computer for controlling the angular displacement of the radiation emitter 7 along the guide track 61. The angular displacement of the radiation source 7 from a central position is in the range of ±20° to ±50°. Advantageously, in this arrangement, there is no requirement for movement of the C-arm 41 in order to take a number of angularly displaced images of the breast as the emitter 7 is simply moved along the arcuate guide track 61. The screen 56 blocks the patient's view of this movement and reduces the anxiety levels for the patient. The emitter 7 is an x-ray source meeting all applicable standards for mammography including but not limited to packaging, collimation, shielding and filtration.

The paddle 11 is mounted on a carriage 73 movable along the guide track 45 by a powered motive assembly. The powered motive assembly is operaply coupled to a computer for controlling the displacement of the paddle 11 along the guide track 45. The biopsy equipment carriage 46 is mounted on a carriage 74 movable along the guide track 45 by a powered motive assembly. The powered motive assembly is also operably coupled to a computer for controlling the displacement of the biopsy equipment carriage 46 along the guide track 45.

Paddle 11 has an aperture 75 for alignment with and allowing passage there through of the biopsy needle 48.

Referring in particular to FIGS. 11 and 12, an extendable arm arrangement 101 is illustrated provided between the mounting bracket 23 and the mammographic head 4. The extendable arm arrangement 101 is provided by an extendable arm 103, 105 provided by two arms 103, 105 having an elbow joint 104. One end of one of the arms 103 has an arcuate swivel plate 102 forming part of the rotary hinge joint 22 coupled to or integrally formed with the balanced arm 3. The end of the other arm 105 distal the elbow joint 104 is designed as an arcuate swivel plate for operable engagement with a mounting bracket 106 having a concave recess formed for receiving the arcuate swivel plate. Advantageously, the elbow joint 104 between the two arms and the rotary hinge joints on each end of the two arms 103, 105 distal the elbow joint 104 further improves the articulation function of the mammographic head 4 relative to the patient's body. The rotary hinge joints 22 have low friction bearings. Advantageously, the low friction bearings help to ensure that the force required to move the mammographic head towards and/or away from the patient's body as a result of patient body movement whilst a breast is clamped are kept well within the range of forces outlined above. The laterally protruding balanced arm 3 comprises a tubular member 111 having one end movably coupled to the collar 16 and the other end movably coupled to the rotary hinge joint 22. The laterally protruding balanced arm 3 comprises a tubular member 11 having one end pivotally coupled to the collar 16 and the other end pivotally coupled to the rotary hinge joint 22 by pivots 120. The laterally protruding arm 3 is a tubular member with a rectangular cross section with each corner of one end pivotally coupled to the collar 16 and each corner of the other end of the tubular member 11 pivotally coupled to the mounting bracket 23 of the rotary hinge joint 22. Advantageously, this allows the laterally protruding balanced arm 3 to move in a vertical plane about the pivotal couplings 120.

Referring to FIG. 13, the balancing arrangement indicated generally by reference numeral 17 of the laterally protruding arm 3 is provided by a pair of gas struts 135, one of which is partly shown. The gas strut 135 extends diagonally along the length of the laterally protruding arm 3 between the ends thereof. The gas struts 135 or other balancing members are mechanically coupled to pivotal couplings 120 at both ends of the balancing arm 3 in a diagonal arrangement. The gas struts 135 are calibrated to support the weight of the balanced arm 3 and the mammographic head 4 at a range of predetermined positions. Advantageously, the adjustable nature of the biasing force in the gas struts 135 allows variable weights of laterally protruding arm 3 and mammographic head 4 combinations to be accommodated. It also provides a means for adjusting the sensitivity of patient movement tracking capability of the mammographic head 4 by adjusting the vertical movement forces required to vertically displace the mammographic head 4.

Referring to FIG. 14, the radiation emitter 7 has three radiation sources 7. The position of each radiation source 7 is adjustable relative to the radiation receiver 8. Advantageously, this allows multiple images of the breast to be taken at a range of angles. Alternatively, the position of the radiation sources 7 is fixed. In a further arrangement, the position of one or more of the plurality of radiation sources 7 is adjustable relative to the radiation receiver 8. Advantageously, this allows multiple images of the breast to be taken at a range of angles. The radiation sources 7 have a switch 161A for switching a single power source between the radiation sources 7. Advantageously, a single power source can supply the predetermined radiation source 7 with power when the specific radiation source 7 is required to image the breast. This reduces the electrical cabling, conduit and space requirements and complexity of design. The switch 161A is a relay or electronic switching arrangement.

Further, the position of the radiation receiver 8 is adjustable within the breast support member 9. The radiation receiver 8 is mounted on a powered motive arrangement 130 which is mounted on a guide track 131 allowing XY movement of the radiation receiver 8 within the plane of the breast support member 9. The powered motive arrangement 130 of the radiation receiver 8 is coupled to the control arrangement of the device 1. The position of the radiation receiver 8 is adjustable in a predetermined way independently and in response to positional adjustment of the radiation emitter 7. Advantageously, this allows a range of imaging techniques to be used where these images are useful to provide different views of the breast tissue.

Referring to the drawings and now to FIG. 15, there is shown a further embodiment of mammographic device indicated generally by the reference numeral 201. A mammographic device 201 having a support member 202 for carrying a mammographic head 203 mounted on the support member 202. The mammographic head 203 having a radiation emitter comprising three radiation source 207. The mammographic head 203 is movably mounted on the support member 202. The plurality of radiation sources 207 comprise a switching arrangement 161A see FIG. 17 for switching a single power source between the three radiation sources 207. Advantageously, a single power source can supply the predetermined radiation source 207 with power when the specific radiation source 207 is required to image the breast. This reduces the electrical cabling, conduit and space requirements and complexity of design. The switching arrangement 161A is a relay and/or an electronic switching arrangement. Switching allows one radiation source 207 to cool down while another is in operation. The radiation source 207 is of the type having a fixed as opposed to rotating anode such as a carbon nanotube x-ray emitter.

The mammographic head 203 further has a radiation receiver 208 and breast clamping assembly 211, 212. The breast clamping assembly 211, 212 has a breast support member 212 for engaging at least a portion of a breast and a compression member 211 for compressing at least a portion of the breast between the support member 212 and the compression member 211. The compression member 211 is movably mounted between the radiation emitter 207 and radiation receiver 208. The breast support member 212 houses the radiation receiver 208. The surface of the breast compression paddle 211 and/or the surface of the breast support member 212 engageable with the patient's breast is provided with an arrangement for increasing friction there between. A cover 215 is provided for the breast compression paddle 211 and/or the breast support member 212. The cover 215 is manufactured from a flexible non-slip material. Advantageously, this increases the friction forces between the breast and the cover 215. The cover 215 or the surface material for the breast compression paddle 211 and/or the breast support member 212 is disposable.

Referring now to FIG. 16 illustrating a further embodiment of the mammographic device of FIG. 15, the mammographic head 203 further has a biopsy arrangement 246 movably mounted on the head 203 between the radiation emitter 207 and the breast clamping arrangement 211, 212. In this second embodiment, the position of the plurality of radiation sources 207 can be fixed. When this configuration is used, the use of fixed position radiation sources 207 means no travel time is required for radiation source 207 travel after a scout image is taken reducing the overall time of the procedure. There is also greater freedom for the source to image distance SID. The risk of any collision between source equipment and any other equipment on the mammography head 203 because of source movement is also eliminated.

This embodiment is also designed to accommodate the position of the three radiation sources 207 being adjustable relative to the radiation receiver 208. Advantageously, this allows multiple images of the breast to be taken at a range of angles. The position of the three radiation sources 207 is determined so that the source to image receptor distance SID is constant for the sources 207. This SID distance is swept out by a radius centred about the location of the image receiver 208.

It will of course be appreciated that this multiple source mammographic device 201 may be used with a support arrangement where patient movement tracking is accommodated as described with reference to the embodiment described in FIGS. 1 to 14. Alternatively, this multiple source mammographic device 201 can be used with a simple support arrangement 202 without patient movement tracking where the support is a portable device such as trolley 2 illustrated in FIG. 1. Furthermore, features of FIG. 16 which are the same as features described with respect to the embodiment of FIG. 14 have retained the same reference numerals.

Referring to the drawings and now to FIG. 17 there is shown a schematic diagram of the components of the overall control system indicated generally by reference numeral 141. An independent control system 143 shown here as a PLC but which could be a bespoke microprocessor system is operably coupled to a paddle motion controller 144a which has a compression driver 144b operably coupled to a compression motor 144c operably coupled to an encoder/position sensor 144d. The encoder/position sensor 144d feeds back information to the paddle motion controller 144a. A radiation emitter motion controller 147a has an emitter driver 147b operably coupled to an emitter motor 147c operably coupled to an encoder/position sensor 147d. The encoder/position sensor 147d feeds back information to the emitter motion controller 147a. A biopsy equipment motion controller 146a-x for controlling the x-axis motion of the biopsy equipment has a biopsy equipment driver 146b-x operably coupled to a biopsy equipment motor 146c-x operably coupled to an encoder/position sensor 146d-x. The encoder/position sensor 146d-x feeds back information to the emitter motion controller 146a-x. A biopsy equipment motion controller 146a-y for controlling the y-axis motion of the biopsy equipment has a biopsy equipment driver 146b-y operably coupled to a biopsy equipment motor 146c-y operably coupled to an encoder/position sensor 146d-y. The encoder/position sensor 146d-y feeds back information to the emitter motion controller 146a-y. A biopsy equipment Z-axis position sensor/encoder 146z provides information in relation to the z-axis position of the biopsy equipment to the PLC 143.

Digital inputs/outputs to the PLC 143 via a digital I/O module 148 comprise safety interlocks 149 for multiple inputs such as access doors. Digital I/O's coupled to fail safe module 148 include optical illumination and PWM control 150, a paddle home position/limit switch input 152, a radiation emitter home position/limit switch input 153, and needle end detection and various needle carrier control inputs/outputs 154. A compression paddle pressure sensor 155 is an analogue input via analogue input/output 156 to the PLC 143. A human machine interface 158 is provided with the PLC 143 to provide a display of a range of information relating to component position, pressure data, radiation emitter status and fault conditions.

A power source 161 for the radiation emitter 162, either single or multiple source, is operably coupled to the PLC 143. The power source 161 having a switching arrangement 161A where a multiple source radiation emitter is utilised. The analog I/O at PLC 143 are current program (0-10V), voltage program (0-10V), voltage monitor (0-10V), current monitor (0-10V) and filament monitor (input 1V=1 A from other control means other than analog control of the emitter power source such as digital control via a serial control interface and protocol are also suitable methods of control). The digital I/O at PLC includes emitter enable, emitter PSU fault and emitter on. The radiation receiver is a flat panel receiver providing the sensitivity and spatial resolution required for this application, our diagram shows a GigE Vision connection to the computer but other camera or non-camera based protocols could be used such as CameraLink or ePCle. A link is also shown from the detector 163 to the Plc 143 as digital I/Os and/or a serial link, the purpose of which is to route control signals through the plc which can provide additional safety features in the event of incorrect operation of other aspects of the system such as failure of normal termination of x-ray radiation. The computer 164 has software for receiving data from the radiation receiver 163 and displaying the data as images in an image display device suitable for use by clinicians. It will of course be appreciated that the PLC 143 can be coupled and decoupled to any one of or any combination of the various controllers to suit the specific design of the equipment.

In relation to the detailed description of the different embodiments of the invention, it will be understood that one or more technical features of one embodiment can be used in combination with one or more technical features of any other embodiment where the transferred use of the one or more technical features would be immediately apparent to a person of ordinary skill in the art to carry out a similar function in a similar way on the other embodiment.

In the preceding discussion of the invention, unless stated to the contrary, the disclosure of alternative values for the upper or lower limit of the permitted range of a parameter, coupled with an indication that one of the said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The features disclosed in the foregoing description or the following drawings, expressed in their specific forms or in terms of a means for performing a disclosed function, or a method or a process of attaining the disclosed result, as appropriate, may separately, or in any combination of such features be utilised for realising the invention in diverse forms thereof as defined in the appended claims.

1.-39. (canceled)

40. A mammographic device, comprising:
a support means for carrying a mammographic head movably mounted on the support means, the mammographic head having a breast clamping means, wherein the mammographic head is movably mounted on the support means so as to be movable in response to forces transmittable through the breast clamping means via a patient's clamped breast as a result of the patient's body movements.

41. The mammographic device as claimed in claim 40, wherein the mammographic head is movable in response to a force less than the force required to slidably move a portion of the patient's clamped breast relative to the breast clamping means.

42. The mammographic device as claimed in claim 40, wherein the mammographic head is movable in a vertical or horizontal direction with the application of a force in a range of 10 Newtons to 75 Newtons.

43. The mammographic device as claimed in claim 40, wherein the breast clamping means comprises a breast support means for engaging at least a portion of the breast and compression means for compressing at least a portion of the patient's breast between the support means and the compression means.

44. The mammographic device as claimed in claim 43, wherein the mammographic head comprises a radiation emitter and radiation receiver.

45. The mammographic device as claimed in claim 44, wherein the radiation emitter comprises a single radiation source.

46. The mammographic device as claimed in claim 45, wherein a position of the single radiation source is adjustable relative to the radiation receiver.

47. The mammographic device as claimed in claim 44, wherein the radiation emitter comprises a plurality of radiation sources.

48. The mammographic device as claimed in claim 47, wherein the plurality of radiation sources comprise switching means for switching a single power source between the plurality of radiation sources.

49. The mammographic device as claimed in claim 48, wherein a position of one or more fixed radiation sources and one or more movable radiation sources is determined so that a source to image receptor distance SID is constant for the fixed radiation sources and through a range of motion of the one or more movable radiation sources, the SID distance being swept out by a radius centered about a location of the radiation receiver.

50. The mammographic device as claimed in claim 49, wherein the radiation emitter is of the type having a fixed as opposed to rotating anode.

51. The mammographic device as claimed in claim 44, wherein compression means are movably mounted between the radiation emitter and radiation receiver.

52. The mammographic device as claimed in claim 44, wherein the breast support means houses the radiation receiver.

53. The mammographic device as claimed in claim 44, wherein a surface of the breast compression means or a surface of the breast support means engageable with the patient's breast is provided with means for increasing friction therebetween.

54. The mammographic device as claimed in claim 40, wherein the mammographic head is freely rotatably movable about the support means or vertically adjustable about the support means.

55. The mammographic device as claimed in claim 40, wherein the support means comprises an upright post having an upright axis.

56. The mammographic device as claimed in claim 40, wherein the support means comprises a support member carrying a laterally protruding arm.

57. The mammographic device as claimed in claim 56, wherein the laterally protruding arm carries the mammographic head.

58. The mammographic device as claimed in claim 56, wherein the laterally protruding arm is freely rotatably movable about the support means or vertically adjustable about the support means.

59. The mammographic device as claimed in claim 40, wherein the support means comprises a balanced arm having balancing means.

60. The mammographic device as claimed in claim 59, wherein the balanced arm is balanced in a wide range of ways comprising an active mechanism having sensors and actuators, mechanical or gas based biasing means, mechanical coupling arrangements, friction, counterweights or any combination of these means or arrangements suitable for providing the balanced arm.

61. The mammographic device as claimed in claim 60, wherein articulation means are provided between an end of the balanced arm distal to the support means and the mammographic head.

62. The mammographic device as claimed in claim 61, wherein the articulation means comprises a rotary hinge joint or a turntable arrangement.

63. The mammographic device as claimed in claim 59, wherein an extendable arm arrangement is provided between the balanced arm and the mammographic head.

64. The mammographic device as claimed in claim 44, wherein the mammographic head has a generally C-shaped arm having a rear face on a spine of the C-shaped arm mechanically coupled to the support means outside a space enclosed by the C-shaped arm.

65. The mammographic device as claimed in claim 64, wherein the C-shaped arm has a front face on the spine defining a guide track for receiving a compression paddle protruding from the guide track into the space enclosed by the C-shaped arm.

66. The mammographic device as claimed in claim 64, wherein the C-shaped arm has a front face on the spine defining a guide track for receiving a biopsy equipment carriage means protruding from the guide track into the space enclosed by the C-shaped arm.

67. The mammographic device as claimed in claim 64, wherein a flange of the C-shaped arm defines the breast support means housing the radiation receiver.

68. The mammographic device as claimed in claim 49, wherein the radiation emitter is movably mounted on guide means which is coupled to the mammographic head.

69. The mammographic device as claimed in claim 68, wherein the guide means is designed so that the source to image receptor distance SID is constant through the range of motion of the one or more movable radiation sources.
